(19) United States
(12) Patent Application Publication
(10) Pub. No.: US 2021/0003605 A1
(43) Pub. Date: Jan. 7, 2021

(54) RETRACTABLE ASSEMBLY, SYSTEM AND METHOD FOR DETECTING MOVEMENT THEREIN

(71) Applicant: Endress+Hauser Conducta GmbH+Co. KG, Gerlingen (DE)

(72) Inventors: Martin Lohmann, Gerlingen (DE); Stefan Pilz, Geithain (DE); Thomas Pfauch, Leipzig (DE); Stefan Paul, Döbeln (DE)

(21) Appl. No.: 16/921,497

(22) Filed: Jul. 6, 2020

(30) Foreign Application Priority Data
Jul. 4, 2019 (DE) 10 2019 118 156.9

(51) Int. Cl. G01P 13/00; G01N 33/00; G01P 15/08

(52) U.S. Cl. CPC G01P 13/00; G01P 15/08; G01N 33/00

(57) ABSTRACT

The present disclosure includes a retractable assembly for immersion, flow and mounted measuring systems in analytical process technology, comprising: an essentially cylindrical housing with a housing interior; a dip tube which can be moved axially in the housing interior between a service position and a process position by means of supply energy, in particular compressed air; and an acceleration sensor. The present disclosure also includes a method for detecting a movement of the dip tube.

### CROSS-REFERENCE TO RELATED APPLICATION

The present application is related to and claims the priority benefit of German Patent Application No. 10 2019 118 156.9, filed on Jul. 4, 2019, the entire contents of which are incorporated herein by reference.

### TECHNICAL FIELD

The present disclosure relates to a retractable assembly, a system comprising the same, and a method for detecting a movement of a dip tube of a retractable assembly.

### BACKGROUND

A great variety of retractable assemblies are offered and marketed by the Endress+Hauser corporate group, for example under the name “Cleanfit CPA875.” Information about them can be found on the Applicant's website, for example, as of the filing date under: http://www.endress.com/cpa875

Retractable assemblies are widely used in analytical measurement technology and process automation. They are used to remove sensors from the process, and thus the medium, without interrupting the process, and to then reintroduce them into the process. The sensors are fastened in a dip tube and are moved axially by hand or automatically, for example pneumatically, by means of a drive between a process position (measurement) and a service position (maintenance, calibration, flushing, probe exchange, etc.). These procedures run within a certain time cycle or as a function of other determinable or measured parameters. The sensors serve to measure one or more physical or chemical process variables.

The field of use of retractable assemblies for measuring physical or chemical process variables of a medium, for example a fluid, in particular a liquid, in process technology is many-faceted. Sensors are used to determine the process variables, wherein the sensors are, for example, pH sensors, conductivity sensors, optical or electrochemical sensors for determining a concentration of a substance contained in the medium to be monitored, such as O₂, certain types of ions, organic compounds, or the like.

If retractable assemblies are used for receiving the sensor for determining at least one process variable, the sensor can be checked, calibrated, cleaned and/or replaced in the service position, wherein the sensor is located in the so-called service chamber. So that the medium is not contaminated by the calibration, flushing or cleaning liquid, the service chamber is sealed off in the service position from the container in which the medium is located in such a way that no exchange of medium/liquid can take place. Usually, for this purpose, a seal is located at the medium-side end of the housing of the retractable assembly and, in interaction with a closure element on the dip tube, prevents the exchange of medium/liquid.

DE102017128888.0, DE102010063970.2 and DE102006061815.7 show retractable assemblies with additional electronics in or on the assembly for performing a diagnosis of the condition of the assembly. The problem to be solved in principle is recognizing that the retractable assembly is imminently stuck due to deposit formation even before failure of the assembly.

In the cited documents, the problem arises that the complexity and the production costs of the assembly increase significantly due to the additional electronics. The energy consumption also increases as a result of such electronics, which leads to significantly increased expenditure during operation at the user, for example as a result of an additional power supply.

### SUMMARY

The present disclosure is based on the object of being able to carry out a diagnosis of the assembly without increasing the costs and impairing operation.

The object is achieved by a retractable assembly comprising an acceleration sensor.

One embodiment provides that the acceleration sensor is arranged in the housing interior.

One embodiment provides that the acceleration sensor is arranged on the dip tube.

One embodiment provides that the retractable assembly comprises at least one insert component, for example, a sensor and/or a cable, wherein the insert component is arranged on or in the dip tube, and wherein the acceleration sensor is arranged on the insert component.

In this embodiment, it is thus advantageous that an additional sensor system is installed not in the assembly, but in the installed cable or in the sensor, the measuring task of which sensor system is not only to detect process oscillations; rather, the focus of it is to detect the movement profiles of the assembly and evaluate and forward them.

One embodiment provides that the retractable assembly comprises a temperature sensor, which is arranged in particular on or in the insert component.

The operating conditions of the assembly can be inferred by measuring secondary parameters, such as the temperature of the sensor or of the cable. At high or low ambient and/or operating temperatures, the assembly is stressed more strongly, or service is needed more frequently.

The object is further achieved by a system comprising a retractable assembly as described above and a transmitter, wherein the transmitter is connected to the acceleration sensor and is designed to detect a movement of the dip tube by means of the acceleration sensor.

The object is further achieved by a method for detecting a movement of the dip tube of a retractable assembly as described above by means of the acceleration sensor.

Some advantages or possibilities result from the fact that the movement of the dip tube can be detected using the acceleration sensor.

The stroke counter of the assembly by means of the acceleration sensor can be used for operation-dependent maintenance.

The travel time between service position and process position can be measured. A change occurring in this time is an early indication of imminently being stuck.

The measurement and evaluation of the movement profile can be used to diagnose the assembly.

The acceleration profile can also give indications of a dirty or insufficiently greased drive and the wear of the seal in the drive.

The quality of the pneumatics (leaks) can be deduced from the movement profile.

If the travel time is too short, high acceleration pulses occur in the end positions. By registering such two parameters, a recommendation for installing or, if present, adjusting a throttle can be given.

If the method is controlled by the transmitter, a control of the movement carried out can take place using the measurement results of the acceleration sensor.

If the stop at the end positions can be clearly detected in the acceleration profile, limit switches on the assembly are no longer necessary. The respective assembly position is supplied to the process controller via the transmitter. This reduces the price of the assembly and eliminates the necessary expenditure for cabling the limit switches.

The integrated acceleration sensor only slightly increases the production costs of the cable or sensor. Changes to the assembly are not necessary. Thus, this solution can be used for all existing assemblies.

A further advantage is that no additional measures are necessary for installation or operation. For example, the user does not have to lay additional power cables or signal cables. Changes are also not necessary in the programming of the process controller, because the travel time of the assembly can be read out as a parameter in the transmitter, for example. A significant change in the travel time could be reported to the controller, for example the process control system, by means of a diagnostic message via a standardized mechanism (for example, NE107).

### BRIEF DESCRIPTION OF THE DRAWINGS

The present disclosure is explained in more detail with reference to the following figures:

FIG. 1 shows the claimed retractable assembly;

FIG. 2 shows the claimed retractable assembly in cross-section; and

FIG. 3 shows the claimed system.

In the figures, the same features are identified with the same reference signs.

### DETAILED DESCRIPTION

“Top,” “above,” and related terms within the meaning of the present disclosure mean facing away from the measuring medium 14. “Bottom,” “below,” and related terms within the meaning of the present disclosure mean facing toward the measuring medium 14.

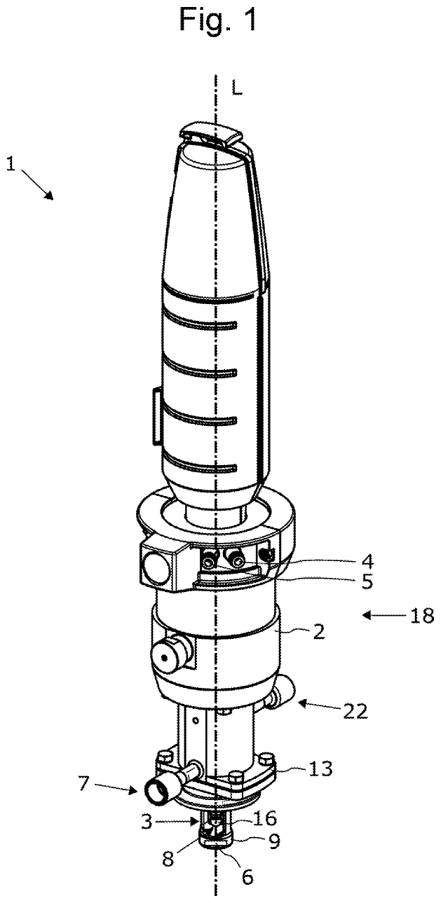

The retractable assembly according to the present disclosure is marked in its entirety with reference sign 1 and is shown in FIG. 1. The retractable assembly 1 consists of an essentially cylindrical housing 2 which can be connected to a container 15 by means of a connecting means 13. The connecting means 13 can be, for example, designed as a flange connection, made of, for example, stainless steel. However, other embodiments are possible. The measuring medium 14 to be measured is located in the container. The container 15 can be, for example, a tank, boiler, tube, pipeline or the like.

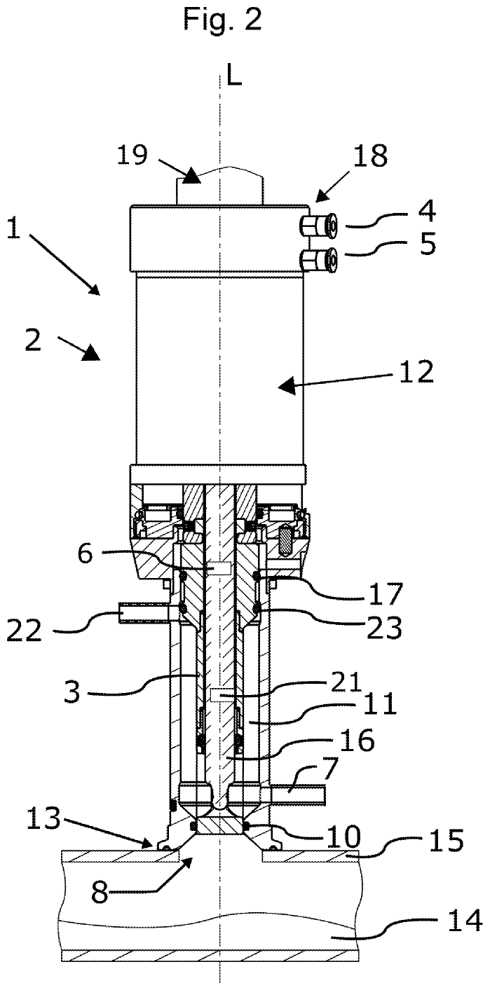

FIG. 1 shows the retractable assembly 1 in the process position. This is explained in more detail below. FIG. 2 shows the retractable assembly 1 in the service position.

A dip tube 3 is guided inside the housing 2. A sensor 16 is connected to the dip tube 3 by a receptacle, not described in greater detail, for example by screwing. The sensor 16 within the meaning of the present disclosure includes sensors for measuring one or more physical or chemical process variables. These are, for example, pH value, also through an ISFET, redox potential, absorption of electromagnetic waves in the measuring medium 14, for example with wavelengths within the UV, IR and/or visible ranges, oxygen, conductivity, turbidity, concentration of metal and/or non-metal materials or temperature. The sensor 16 has access to the measuring medium 14 through an opening 8 in the dip tube 3. In this case, the opening 8 is designed in such a way that it is open in the flow direction, that is, that the measuring medium 14 optimally flows to the sensor 16, in particular if the retractable assembly 1 is used in a pipeline.

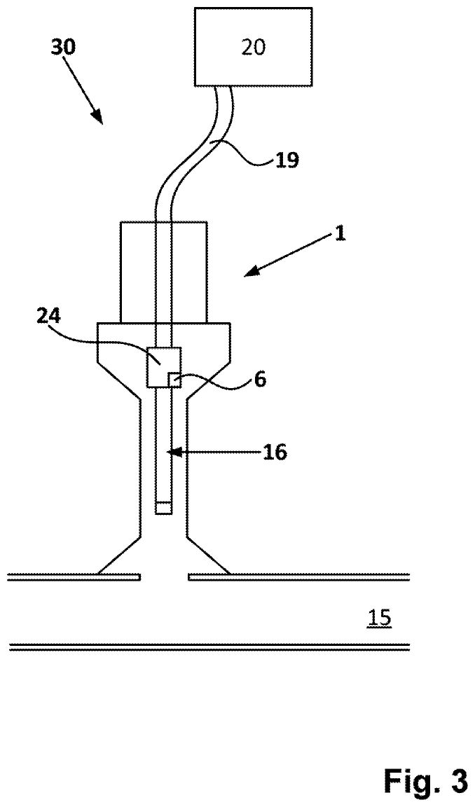

The sensor 16 is connected to a cable 19, see also FIG. 3 in this respect. The cable in turn is connected to a transmitter 20. A system 30 comprises a retractable assembly 1, a sensor 16, a cable 19 and a transmitter 30, see also FIG. 3. Within the meaning of the present disclosure, sensor 16 and cable 19 are an “insert component.” The insert component is located at least in sections in the housing interior 12.

The dip tube 3 can be produced from various materials. The prior art is aware of dip tubes 3 made of steel or stainless steel. However, applications, in which highly resistive materials are used, are common, in particular in the chemical industry. The dip tube 3 can thus also be made of a plastic, such as polyether ether ketone (PEEK), polytetrafluorethylene (PTFA), a perfluoroalkoxy polymer (PFA), another plastic or resistant metals, such as Hastelloy. The same is true for the housing 2.

The dip tube 3 is mounted in a manner axially displaceable in the direction of the measuring medium 14 or in the direction facing away from the measuring medium 14 along the central axis L. The dip tube 3 can be moved between the service position (shown in FIG. 2) retracted into the housing 2 and the process position (shown in FIG. 1) extended out of the housing 2. The measurement takes place in the process position. The probe or sensor 16 has access to the measuring medium 14 through a cage-like opening 8 in the dip tube 3. A wide variety of service tasks, such as cleaning or calibration, are performed in the service position. Flushing/cleaning/calibration and/or sterilization medium can be admitted into the service chamber 11 through the connection 7, see below). In general, flushing/cleaning/calibration and/or sterilization medium are to be referred to herein as “medium.” The medium can be liquid or gaseous. The liquid can drain again through the corresponding connection 22, which can be positioned in a manner both axially and radially offset from the connection 7. The flushing direction can also be reversed.

The displacement of the dip tube 3 is performed by a drive device 18 located above the service chamber 11. The drive 18 is part of the housing 2. The housing 2 comprises a housing interior 12. The movement is carried out by an automatic drive, for example by supply energy. If supply energy is introduced through the connection 4, the dip tube 3 moves from the service position into the process position. The connection 5 then serves as an outlet. If supply energy is introduced through the connection 5, the dip tube 3 moves from the process position into the service position. The connection 4 then serves as an outlet. Pneumatic, hydraulic or electric drives, for example, are known from the prior art. The present retractable assembly uses a pneumatic drive. The process of displacing the dip tube 3 is explained in more detail below.

A piston (not shown) is firmly connected to or an integral part of the dip tube 3. The piston is designed, for example, as an annular piston and forms part of the drive 18. The piston divides the drive portion of the housing interior 12 into an upper region and a lower region. The dip tube 3 can be moved through the connection 4 into the upper region and through a connection 5 into the lower region above or below the piston: When compressed air is brought into the upper region through the connection 4, the dip tube 3 moves in the direction of the medium 14, wherein air from the lower region simultaneously flows through the connection 5. Air can also be actively drawn from the lower region in order to support the movement in the direction of the medium 14. When compressed air is brought into the lower region through the connection 5, the dip tube 3 travels away from the medium 14, wherein air from the upper region simultaneously flows through the connection 4. Air can also be actively drawn from the upper region in order to support the movement.

It is self-evident that corresponding seals (not shown) need to ensure that compressed air does not escape and is only conducted through the connections 4, 5.

The connections 4, 5 are attached laterally to the housing 2. The connection 4 can be located above the piston (dip tube 3 in service position), the connection 5 can be located below the piston (dip tube 4 in process position). It is conceivable for both inlets 4, 5 to be located on the housing 2 above or below the piston and, for proper functioning, for a line to be guided into the respective other region in the interior of the housing 2. FIG. 1 shows that connections 4, 5 are arranged next to one another above the piston (service position). FIG. 2 shows them arranged one above the other. A corresponding line for guiding the connection 5 into the lower region is located in the interior of the housing 2. The connections 4, 5 do not necessarily have to be located in the same frontal plane.

If the dip tube 3 is in the service position, a portion of the dip tube 3, in particular the sensor 16, is located in the service chamber 11 for flushing, cleaning, calibrating, sterilizing, etc. The closure element 9 for process separation is located at the lower end of the dip tube 3. The closure element 9 seals off the service chamber 11 from the process, and thus from the measuring medium 14. The measuring medium may be hot, toxic, caustic or otherwise harmful to humans and the environment. It must therefore be ensured that the closure element 9 seals reliably and permanently. For this purpose, various sealing devices are attached to the housing 2; in particular, one or more medium seals 10 are used. In the depicted embodiment, the medium seal 10 is arranged on the housing 2. Alternatively, the medium seal 10 may be arranged on the lower end region of the dip tube 3 (not shown).

At least one seal 17, in the example two seals 17, 23, is arranged on the upper region of the dip tube 3. The seal 17, 23 seals off the service chamber 11 from the drive device 18, particularly when moving from the service position to the process position and vice versa. In the service position, the upper seal 17 is arranged above the connection 22 and the lower seal 23 is arranged at the same height or below the connection 22.

The retractable assembly 1 comprises an acceleration sensor 6.

There are various ways of arranging the acceleration sensor 6. In principle, the acceleration sensor 6 is arranged in the housing interior.

In one embodiment, the acceleration sensor 6 is arranged on the dip tube 3 or, more generally, on a movable part of the retractable assembly 1. This is illustrated in FIG. 1.

In an advantageous embodiment, however, the acceleration sensor 6 is arranged on the insert component, that is, on sensor 16 or cable 19. FIG. 2 shows an embodiment in which the acceleration sensor 6 is arranged on the sensor 16. It is arranged approximately in the central region. The acceleration sensor 16 can also be arranged on the upper region of the sensor 16. This is the case, in particular if the sensor 16 is of the “Memosens” type of the applicant. In this case, but also in principle, the acceleration sensor 16 can be arranged in the sensor head 24, that is, that part of the sensor 16 which is designed for further connection, for example to the cable 19.

A cable end, so to speak, a type of “cable connection dummy,” that is, a placeholder, can also be attached to the assembly 1 as an insert component. As a result, such a cable with acceleration sensor 16 can be connected to the assembly 1 purely as a movement sensor. A direct coupling from cable to assembly is thus achieved.

The retractable assembly 1, in particular the sensor 16 (see FIG. 2), comprises a temperature sensor 21.

FIG. 3 shows a system 30 comprising a retractable assembly 1 and a transmitter 20, wherein the transmitter 20 is connected to the acceleration sensor 6 via a cable. The transmitter 20 is designed to detect a movement of the dip tube 3 by means of the acceleration sensor 6.

This gives rise to the possibility of detecting the movement of the dip tube 3 of the retractable assembly 1 by means of the acceleration sensor 6.

The transmitter 20 thus comprises evaluation electronics with software which calculates from the sensor signals of the acceleration sensor 6 the movements of the assembly 1, that is, for example, the number, duration, speed or acceleration of the stroke movement from the service position and measurement position and vice versa. Diagnostic information for the assembly 1 can then ultimately be calculated therefrom.

1. A retractable assembly for immersion, flow and mounted measuring systems in analytical process technology, the retractable assembly comprising
a substantially cylindrical housing that defines a housing interior;
a dip tube that is axially movable in the housing interior between a service position and a process position using supply energy; and
an acceleration sensor.

2. The retractable assembly of claim 1, wherein the supply energy is compressed air.

3. The retractable assembly of claim 1, wherein the acceleration sensor is disposed in the housing interior.

4. The retractable assembly of claim 1, wherein the acceleration sensor is disposed on the dip tube.

5. The retractable assembly of claim 1, wherein:
the retractable assembly further comprises at least one insert component;
the insert component is disposed on or in the dip tube; and
the acceleration sensor is arranged on the insert component.

6. The retractable assembly of claim 5, wherein the at least one insert component is a sensor and/or a cable.

7. The retractable assembly of claim 1, wherein the retractable assembly further comprises a temperature sensor, which is disposed on or in the insert component.

8. A system, comprising:
a retractable assembly according to claim 1; and
a transmitter, wherein the transmitter is connected to the acceleration sensor and is configured to detect a movement of the dip tube using the acceleration sensor.

9. A method for detecting a movement of the dip tube of a retractable assembly, the method comprising:
providing a retractable assembly according to claim 1; and
detecting a movement of the dip tube via the acceleration sensor using a transmitter, wherein the transmitter is connected to the acceleration sensor and is configured to detect a movement of the dip tube via the acceleration sensor.
